(19) United States
(12) Patent Application Publication
(10) Pub. No.: US 2003/0077841 A1
(43) Pub. Date: Apr. 24, 2003

(54) Therapeutic potential of immunoglobulin preparations

(76) Inventors: Jean-Pierre Bouvet, Paris Cedex (FR); Srini V. Kaveri, Paris Cedex (FR); Michel D. Kazatchkine, Paris Cedex (FR); Peter Spath, Bern (CH)

Correspondence Address: SUTHERLAND ASBILL & BRENNAN LLP, 999 PEACHTREE STREET, N.E., ATLANTA, GA, 30309, US

(21) Appl. No.: 10/149,896

(22) PCT Filed: Dec. 13, 2000
(86) PCT No.: PCT/EP00/12670

(30) Foreign Application Priority Data
Dec. 13, 1999 (EP) 99124769.3

(51) Int. Cl. G01N 33/543; C07K 16/00

(52) U.S. Cl. 436/518; 530/387.1

(57) ABSTRACT

The present invention refers to a method of enhancing the activity of an immunoglobulin preparation and to immunoglobulin preparations obtainable by said method.

The present invention refers to a method of enhancing the activity of an immunoglobulin preparation and to immunoglobulin preparations obtainable by said method.

Human immunoglobulins, particularly immunoglobulins of the class G (IgG) are used increasingly as therapeutics for the treatment of various diseases: their first use was as replacement therapy in case of primary immunodeficiency; subsequently, they were used in a number of other conditions, e.g. in idiopathic thrombocytopenic purpura (ITP), pediatric acquired immunodeficiency syndrome (AIDS), systemic lupus erythematodes, Guillain-Barré-syndrome, and other neurological diseases. The therapeutic application therefore makes use of the antigen-binding functions of the molecule for binding to foreign antigens, as well as of its Fc-functions for immunomodulation. The antigen-binding functions are part of the variable portion of the molecule, while the Fc-functions reside on the constant domains. The multiplicity of antigens humans are faced with induces a corresponding multitude of antibodies that are made by the immune system in response to the antigens. It is estimated that a single individual produces as many as 10⁶ different antibodies, a population of human blood donors may produce as many as 10⁹ different specificities.

Immunoglobulins, particularly IgGs, occur in soluble form in human plasma and are one of the most prominent classes of plasma proteins. They can be isolated by different techniques from human plasma. Industrial production of immunoglobulins starts with pooled human plasma, which has been obtained either by centrifugation of donated, whole blood (so-called recovered plasma), or by drawing only plasma from donors by a process called plasmapheresis (this type of plasma is called source plasma). In either case, the starting pool contains a large number of individual donations and a correspondingly large number of immunoglobulin specificities.

Several methods of plasma fractionation are known; the goal of every one of them is to produce immunoglobulin in optimal yield, sufficient purity, proven safety, and at a reasonable cost. Although in times past the best known method for protein fractionation in general was precipitation with ammonium sulfate, the most used precipitation agent for commercial fractionation of human plasma is ethanol. Cohn and his collaborators at Harvard University established, during World War II, a fractionation scheme based on the five variables ethanol, protein and salt concentration, pH-value, and temperature, which is still used today, either in its original form or as variants. Other possibilities are also explored, e.g. isolation of plasma proteins by various types of chromatography, by membrane filtration, by electrophoresis, or combinations of different methods.

It is one of the primary goals of all present fractionation methods to leave the proteins intact, i.e. not to introduce any modifications of the covalent or non-covalent structure, including the secondary, tertiary and quaternary structure of the proteins. This is important, because the currently available scientific evidence clearly demonstrates that intactness of the proteins is a prerequisite for their biological function. It can indeed easily be shown that, e.g., enzymes loose their activity when they are subjected to treatments like heat or chaotropic agents which destroy their three-dimensional structure. In some cases, the original three-dimensional structure may be recovered, and with it the corresponding biological activity. For instance, an enzyme may be rendered inactive by dissolving it in 6 molar urea; activity is often restored after dialyzing out the urea. Covalent modifications, on the other hand, usually result in permanent loss of activity. In some cases, enzymes may also be re-engineered by systematic and rational changes of some amino acids that are located close to the active site; the new proteins may have improved, altered or diminished catalytic activities.

Vincent and Revillard (Mol. Immunol. 20 (1983), 877-884) describe an increased binding activity of serum IgG after treatment of serum components by dissociating buffers. It is assumed that this increased activity is due to dissociation of antigen complexed in the serum. Maeda et al. (Protein Eng. 9 (1996), 95-100) disclose an effective renaturation of denatured and reduced IgG in vitro without assistance of chaperones. An enhanced antigen binding activity of the renatured immunoglobulin preparation is not disclosed.

According to the present invention, it was surprisingly found that it is possible to either improve pre-existing or even induce previously absent activity, particularly antigen-binding activity, in immunoglobulins by subjecting them to denaturation, e.g. by chaotropic agents, followed by restoration of their structure after removal of these agents by a suitable process, e.g. dialysis. In view of the state of the art, this discovery was totally unexpected. The treated immunoglobulins may be used as pharmaceuticals with improved properties. An increased activity will also result in smaller amounts of immunoglobulin being needed for treatment; this will help to alleviate the current shortage of these pharmaceuticals. The treated immunoglobulins may also be used as reagents for diagnostic assays, particularly for the immunological determination of analytes in a sample, e.g. in a biological sample.

A subject matter of the present invention is a method for enhancing the activity of an immunoglobulin preparation comprising at least one step of denaturing and subsequently renaturing the immunoglobulin.

The immunoglobulin is preferably a glycosylated immunoglobulin as obtainable from eukaryotic host organisms or host cells. The immunoglobulin may be selected from polyclonal antibodies, monoclonal antibodies and fragments thereof containing antigen-binding determinants, e.g. F(ab′)₂ fragments. Further, the present invention also encompasses recombinant immunoglobulins such as single chain antibodies or hetero-bispecific antibodies. More particularly, the immunoglobulin is selected from IgG antibodies and fragments thereof.

Immunoglobulin preparations may be obtained by different methods. Polyclonal immunoglobulin preparations may be obtained from individual donors, e.g. from healthy blood donors or from patients, e.g. myeloma patients, or from plasma pools. For the isolation of immunoglobulin on a laboratory scale precipitation with ammonium sulfate may be used. Myeloma patients produce large amounts of so-called paraprotein, which is a natural, monoclonal IgG. Commercially available IgG preparations for intravenous application may also be used, either as delivered to the patient, or after enzymatic modification, e.g. as F(ab′)₂ fragments after cleavage with pepsin. On the other hand, immunoglobulin preparations may also be obtained from non-human animals, from hybridoma cells (monoclonal antibodies) or from other eukaryotic host cells, e.g. Chinese hamster ovary (CHO) cells (recombinant immunoglobulins).

The immunoglobulin preparation may be obtained from any known source in a conventional manner. The invention resides in a treatment step wherein the immunoglobulin is subjected to at least one denaturing/renaturing procedure. The denaturing/renaturing procedure may be carried out at any stage of the purification protocol which is used for the manufacture of an immunoglobulin preparation, particularly an immunoglobulin preparation for pharmaceutical purposes. Preferably, the denaturing/renaturing procedure is carried out with a substantially purified immunoglobulin preparation. The immunoglobulin preparation to be treated is preferably free from substantial amounts of immunoglobulin-bound antigen.

The denaturing/renaturing procedure according to the present invention may comprise:

(a) treating the immunoglobulin preparation with a chaotropic agent and subsequent removal of the chaotropic agent by suitable means, e.g. dialysis, diafiltration, chromatography etc.,

(b) treating the immunoglobulin preparation at a low pH, particularly at a pH from 1.5 to 2, more particularly at a pH of about 2, for a sufficient time period of e.g. 0.5 h and subsequent readjusting the pH to approximately neutral,

(c) treating the immunoglobulin preparation at a high pH, particularly at a pH from 10 to 11.5, for a sufficient time period of e.g. 0.5 h and subsequent readjusting of the pH to approximately neutral, and

(d) any combinations of at least two of (a), (b) and (c), particularly combinations of at least (a) and (b) or (a) and (c).

According to the above embodiments, a chaotropic agent and/or an acid are used in an amount which is sufficient to accomplish denaturation of the immunoglobulins contained in said immunoglobulin preparation. Preferably, the denaturation is carried out gradually, i.e. the amount of chaotropic agent and/or the pH are adjusted in such a manner that at first a partial denaturation occurs. Then, by further adding chaotropic agent and/or lowering or raising the pH, the denaturation of the immunoglobulins may be completed.

The chaotropic agent may be selected from urea, guanidine and guanidinium salts, e.g. guanidinium hydrochloride, thiocyanates, e.g. ammonium thiocyanate or alkali thiocyanates such as sodium thiocyanate, iodides, perchlorates and combinations thereof. It should be noted, however, that other chaotropic agents are also suitable for the purpose of the present invention. The removal of the denaturing agent is preferably carried out by dialysis against suitable buffers.

It is obvious for the skilled person that variants of the basic process as described above, e.g. with other chaotropic agents and/or other ways to introduce and/or remove said chaotropic agents and even other ways to influence the structure of the immunoglobulin molecules may be designed. The concept of the present invention is the improvement of the functionality of a protein by modulating its structure through one or several denaturation/renaturation cycles. This is the general teaching of this patent application and it is, as such, not even limited to immunoglobulins, but may be extended to other proteins, e.g. enzymes.

A further subject matter of the present invention is an immunoglobulin preparation obtainable by the method as described above. The immunoglobulin preparation of the invention has an enhanced functionality, particularly an enhanced antigen-binding activity compared to an untreated immunoglobulin preparation. Preferably, the antigen-binding activity is at least 1.5 times higher or preferably at least 2 times higher than the antigen-binding activity of the immunoglobulin preparation in untreated form, wherein the activity is determined at an immunoglobulin concentration of the treated preparation, at which the treated preparation has about 50% of the highest activity obtainable by said treatment. Assays of the activity of untreated and treated preparations may be done with standard procedures available in biochemistry laboratories, particularly, the activity of immunoglobulin preparations may be carried out by ELISA assays coated with corresponding antigens. The binding of immunoglobulins to said antigens may be detected with labeled rabbit and goat antibodies which have been raised against human Fcγ and F(ab′)₂ fragments. Binding inhibition may be measured by incubation of immunoglobulins with solutions of the relevant antigen prior to the ELISA assay.

The present invention is further explained in detail by the following figures and examples.

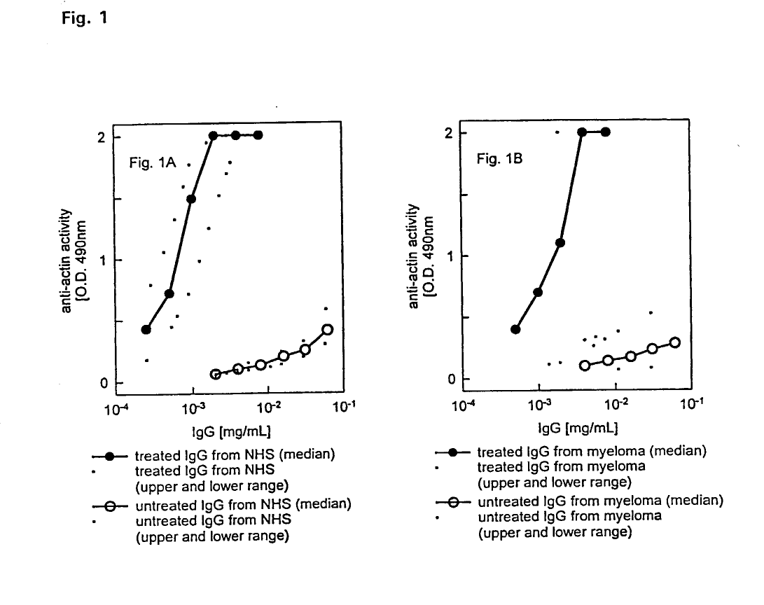

FIG. 1 shows an increase in antigen (actin)-binding activity by treating immunoglobulins from normal serums (NHS) from healthy donors (A) and from myeloma patients (B) with chaotropic agents,

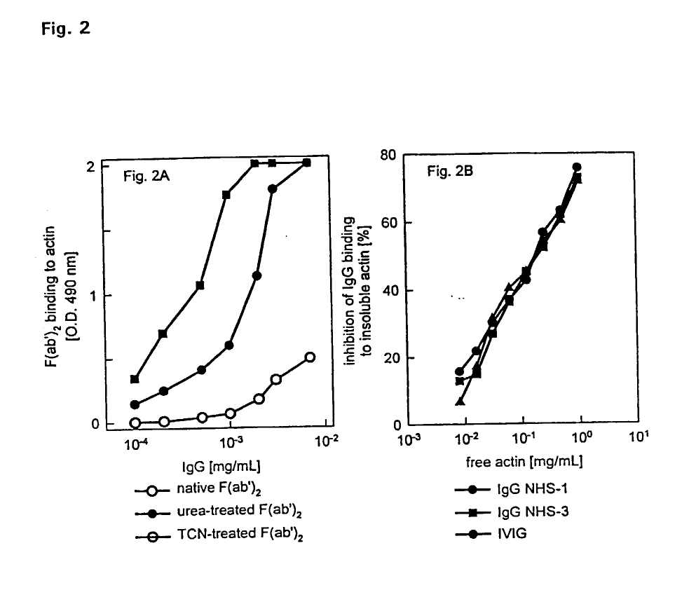

FIG. 2 shows the antigen-binding activity of native and treated F(ab′)₂ preparations (A) and the inhibition of IgG binding to insoluble antigen (B),

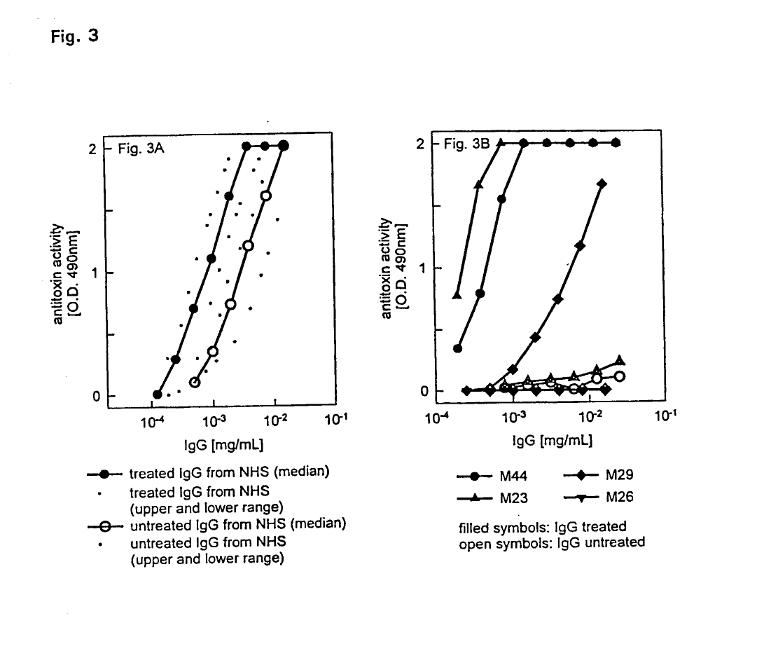

FIG. 3 shows an increase in antigen (toxin)-binding activity by treating IgG preparations with chaotropic agents,

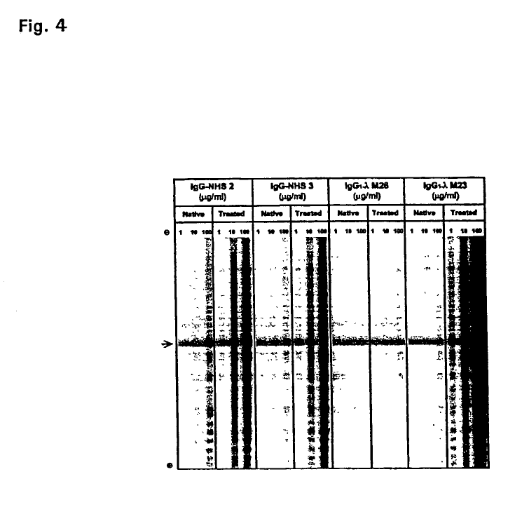

FIG. 4 shows an increase in antigen (human liver protein)-binding activity by treating IgG preparations with chaotropic agents,

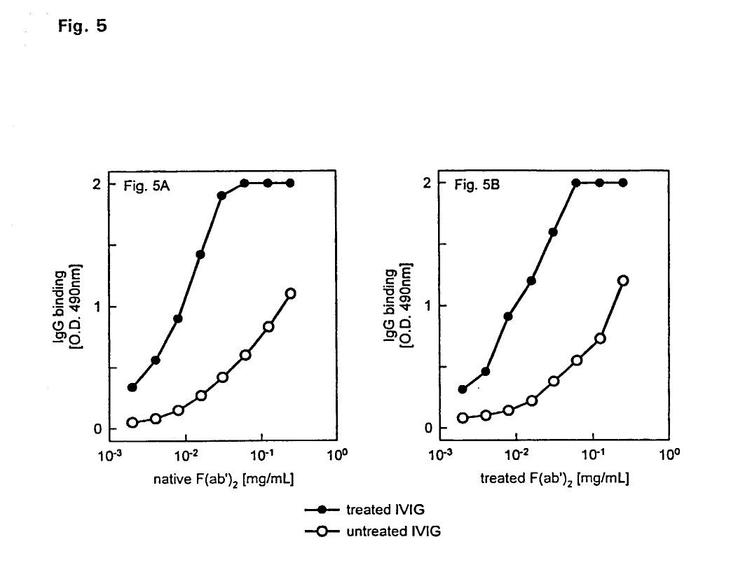

FIG. 5 shows an increase in antiidiotypic activity by treating IgG preparations with chaotropic agents, and

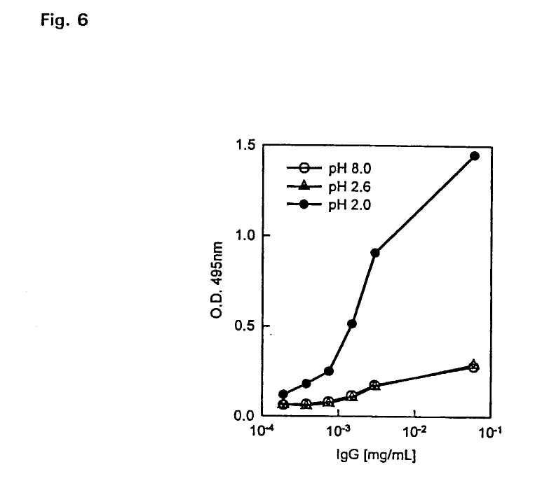

FIG. 6 shows the effect of pH-treatment on antigen (actin)-binding activity of IgG preparations.

### EXAMPLES

The following immunoglobulin preparations were subjected to treatment with chaotropic agents:

polyclonal IgG from single donation

monoclonal IgG (myeloma protein) from single donation

polyclonal IgG from plasma pool

polyclonal F(ab′)₂ from plasma pool

These products were treated in the following way: aqueous solutions of the individual products, containing about 1 mg/mL of protein in phosphate-buffered saline (PBS), were placed in dialysis bags and dialyzed against a 6-molar solution of urea in PBS or against a 1.3-molar solution of sodium thiocyanate (NaSCN) in PBS. In order to achieve a gradual denaturation of the proteins dialysis was carried out at first against a 3-fold volume excess (compared with the content of the bag) of either urea or NaSCN, then against a 50-fold excess volume of the same reagent. After completion of this treatment, the denaturing reagents were removed by another dialysis step, this time against PBS without the agents. The renatured proteins were stored until use at 4° C. in PBS in the presence of 50% v/v of glycerol.

### Example 1

Eleven different IgGs prepared from single plasma units were assayed for their binding activity to immobilized actin either before or after treatment with 6 M urea. All of them showed increasing bindung after treatment, with little variation from one preparation to the other (FIG. 1A). A similar experiment was carried out with myeloma IgG from 10 different patients; in this case, there were differences from one preparation to another; while there was only a comparatively small increase in binding to actin in some cases, there was tremendous enhancement in others (FIG. 1B).

### Example 2

F(ab′)₂ fragments were prepared from a therapeutic, polyclonal IgG and their binding to immobilized actin assessed either without treatment or after treatment with 6 M urea or 1.3 M sodium thiocyanate (TCN). Binding was markedly enhanced after treatment with either one of the chaotropic agents, with thiocyanate even more than with urea (FIG. 2A). Further, pre-incubation of urea-treated IgG of NHS-1 and NHS-3 and of IVIG with soluble actin inhibited the reactivity of treated IgG with actin. These results indicate that the interaction of treated IgG with actin is indeed specific (FIG. 2B).

### Example 3

Binding of therapeutic, polyclonal IgG to tetanus toxoid was measured with a microtiter plate assay. There was only a comparatively small increase in binding activity when 6 M urea-treated IgG was compared to identical, untreated IgG (FIG. 3A). Monoclonal IgG, on the other hand, obtained from myeloma patients, behaved very differently, depending on the patient they were obtained from. Some myeloma proteins (e.g. M26) did not demonstrate any increase in binding activity after treatment with 6 M urea. Others (e.g. M29, M44 and M23) showed moderately to very strongly increased binding activity (FIG. 3B).

### Example 4

Increased binding of 6 M urea-treated IgG was also demonstrated against immobilized proteins from human liver. Liver proteins were separated by SDS-PAGE and transferred to nitrocellulose filters. The filters were reacted with the IgG to be tested; bound IgG was revealed with a labeled second antibody. As shown in FIG. 4, binding of IgG isolated from two normal subjects (IgG-NHS-2 and IgG-NHS-3) to liver proteins was greatly enhanced after treatment with 6 M urea. Of the two myeloma proteins tested, the one which did not show increased binding to tetanus toxoid (M26) demonstrated only a small increase in reactivity to liver proteins after urea treatment; M23, on the other hand, whose binding to tetanus toxoid could be enhanced by urea treatment, also demonstrated greatly increased binding to liver proteins after urea treatment.

### Example 5

Untreated and urea-treated, polyclonal IgG (IVIG) was reacted with immobilized F(ab′)₂ fragments which themselves had either not been treated or had been treated with 6 M urea. Binding was increased when urea-treated IgG was used, as compared to untreated IgG. Treatment of the F(ab′)₂ fragments, on the other hand, had no influence on binding of IgG (FIG. 5A and B). Urea treatment selectively increases the anti-idiotypic activity.

### Example 6

IgG prepared from serum of a healthy donor was diluted with distilled water; the pH was brought to either 2 or 2.6 with 0.1 M Soerensen buffer and the solution was incubated for 30 min at room temperature. After incubation, the pH was readjusted to 8 with Tris-HCl-buffer, and salts were reduced by dialysis against PBS. The control was incubated with PBS instead of Soerensen buffer. The binding activity of the preparations against human actin was then measured. As shown in FIG. 6, this activity was greatly increased only in the preparation which had been incubated at pH 2, but not in any of the other samples.

1. A method for enhancing the activity of an immunoglobulin preparation, comprising at least one step of denaturing and subsequently renaturing the immunoglobulin.

2. The method of claim 1,
wherein said immunoglobulin is selected from polyclonal antibodies, monoclonal antibodies and fragments thereof containing antigen-binding determinants.

3. The method of claim 2,
wherein said antibodies are IgG antibodies.

4. The method of any one of the preceding claims,
wherein the denaturing/renaturing step is selected from
(a) treating said preparation with a chaotropic agent and subsequent removal of the chaotropic agent,
(b) treating said preparation at a low pH and subsequent readjusting the pH to approximately neutral,
(c) treating said preparation at a low pH and subsequent readjusting the pH to approximately neutral, and
(d) combinations of at least two of (a), (b) and (c).

5. The method of claim 4,
wherein the chaotropic agent is selected from urea, guanidine and guanidinium salts, thiocyanates and combinations thereof.

6. The method of any one of the preceding claims, wherein the antigen binding activity of the immunoglobulin preparation is enhanced.

7. An immunoglobulin preparation obtainable by the method of any one of claims 1 to 6.

8. The immunoglobulin preparation of claim 7, having an antigen binding activity which is at least 1.5 higher than the antigen binding activity of an untreated immunoglobulin preparation.

9. The immunoglobulin preparation of claim 7 or 8, which is a pharmaceutical preparation.

10. The immunoglobulin preparation of claims 7-9 for therapeutic or diagnostic use.
